(19) United States
(12) Patent Application Publication
(10) Pub. No.: US 2022/0157195 A1
(43) Pub. Date: May 19, 2022

(54) Systems and Methods for a Simulator for Brain Mapping

(71) Applicant: Mayo Foundation for Medical Education and Research, Rochester, MN (US)

(72) Inventors: Rebecca B. Forry, Parker, CO (US); Fidel Valero-Moreno, Jacksonville, FL (US); Maite S. Marin-Mera, Viejo, TX (US); Faith T. Colaguori, Tampa, FL (US); Jaime L. Martinez Santos, Charleston, SC (US); Megan E. McDonnell, Holland, PA (US); W. C. Fox, Jacksonville Beach, FL (US); Karim ReFaey, Jacksonsville, FL (US); Alfredo Quinones-Hinojosa, Ponte Vedra Beach, FL (US); William E. Clifton, III, Jacksonville, FL (US); Aaron C. Damon, Jacksonville, FL (US)

(21) Appl. No.: 17/524,496

(22) Filed: Nov. 11, 2021

Related U.S. Application Data
(60) Provisional application No. 63/115,738, filed on Nov. 19, 2020.

(51) Int. Cl. G09B 23/30; A61N 1/05

(52) U.S. Cl. CPC G09B 23/30; A61N 1/0551

(57) ABSTRACT

The present disclosure provides a surgical training model apparatus and a method for creating a surgical training model. The training model apparatus includes a functional brain model that responds to electrical stimulation and enables users to simulate cortical brain mapping outside the operating room. Methods for creating a surgical training model include consideration of engineering design inputs and other parameters.

### CROSS-REFERENCES TO RELATED APPLICATIONS

This application claims the benefit of U.S. Provisional Patent Application Ser. No. 63/115,738 filed on Nov. 19, 2020 and entitled “Simulator for Brain Mapping,” which is incorporated herein by reference as if set forth in its entirety for all purposes.

### STATEMENT REGARDING FEDERALLY SPONSORED RESEARCH

Not Applicable.

### BACKGROUND OF THE INVENTION

### 1. Field of the Invention

This invention relates to a surgical simulation and training model apparatus and methods for making a surgical simulation and training model.

### 2. Description of the Related Art

Almost 60,000 invasive brain tumor removal surgeries take place every year in the United States and Canada. These neurosurgical procedures are advanced and require extensive training, typically done during a 7-year residency program, which is the longest medical residency. There are several thousand surgical residents in the United States who primarily learn to perform these difficult procedures through observation of current neurosurgeons and participation in cadaver labs. Cadavers are fairly accessible in the United States but can cost up to USD $2,500.00 and imply some ethical and religious concerns. Additionally, there are many practical issues with this approach.

The current gold standard in neuroncolytic tumor resection surgery training involves anatomical and practical labs conducted on cadaver brains. Cadaver brains, however, are fixed and provide several obstacles to accurate modeling, including inaccuracies in material properties and the inability to respond to electrical stimulation. In modern neurosurgical practice, intraoperative brain mapping, through direct electrical stimulation of cortical and subcortical structures, constitutes a critical component of awake tumor resection surgery that helps neurosurgeons avoid cutting or damaging essential cortical elements and subcortical pathways. Stimulation is traditionally applied via a bipolar electrode in a technique that neurosurgery residents learn through observation in the operating room.

While training for neurosurgical procedures on a cadaver can help a trainee go through the motions of a procedure, as noted there are many important features of a live brain surgery that do not translate to a cadaver. One of these features is the texture of the brain tissue, and another is that a user cannot train tumor resection. Cadaver tissue is fixed, meaning that it was filled with chemicals to preserve it. This introduces a vast difference in texture and material density between the fixed and live brain tissue, which makes training on the cadaver less accurate to true surgical operations. Additionally, the cadaver tissue does not bleed or pulse with a heartbeat like live tissue does. Finally, and perhaps most importantly, the cadaver tissue is unable to respond to electrical stimulation like a live brain, which is a crucial part of brain tumor resection surgery.

Functional brain mapping was introduced over a century ago and is based upon the principle of nerve conduction. Currently, this technique is widely used in neurosurgery with applications including neuro-oncology, epilepsy surgery, and surgery of selective vascular lesions. Functional brain mapping represents the most reliable tool for identifying and protecting eloquent brain tissue, including speech and motor tracts, during surgery. Mastering the technique of brain mapping requires rigorous training including live observation and operation, which is complicated by changing expectations of attending physicians and reduced resident exposure and autonomy due to the recent COVID-19 pandemic. Surgical education through simulation may reduce the learning curve and error rates, increase resident autonomy, and increase patient safety and improve patient outcomes.

In order to combat some of these challenges, a new type of device is being considered to train medical professionals to perform tumor resection surgeries: functional anatomical models. However, the current offering of products in this field is primitive and, despite the goals, non-functional. There are several technological challenges in creating a functional model. One of these challenges involves the selection of material for the model. Live brain tissue is quite soft and flexible, unlike many synthetic materials used for modeling. The challenge lies in finding a material that can more effectively mimic the texture of live brain tissue. While a true model would incorporate all the aspects of the live brain, much of the brain is still misunderstood and therefore difficult to model. Another current challenge is to scale back the number of critical components or anatomical considerations in order to create the first iteration of a functional model.

The current training methods for neurosurgery procedures lack important aspects of live tumor resection surgery, including simulation of real-time electrical feedback, which is critical for procedure success. What is needed, therefore, is an improved surgical training model apparatus and methods for creating a surgical training model apparatus.

### SUMMARY OF THE INVENTION

Systems and methods are provided for a training apparatus that includes a functional brain model that responds to electrical stimulation and enables users to simulate cortical brain mapping outside the operating room. Methods for creating a surgical training model include consideration of engineering design inputs, such as metrics in electrical stimulation performance, elasticity, brain tract inclusions, anatomical accuracy, low-grade glioma pathophysiology, product shelf life, and the like. The model may be used by practicing neurosurgeons, neurosurgery residents, and the like.

In one aspect, a surgical training model apparatus is provided. The surgical training model apparatus includes a body that emulates brain tissue and a wire that emulates a corticospinal tract, the wire being at least partially surrounded by the body.

In some aspects, the surgical training model includes a component that emulates a tumor that is at least partially surrounded by the body. In some aspects, the body has a first color, the component has a second color, which is different from the first color. In some aspects, the body has a transition region adjacent to the component that has a color gradient between the first color and the second color.

In some aspects, the body comprises a polymeric material. The polymeric material may have an elasticity of 7000 kPa±440 kPa. The polymeric material may be selected from polyvinyl alcohol, polylactic acid, silicones, thermoplastic elastomers, and mixtures and blends thereof.

In some aspects, the wire used in the surgical training model has a diameter in a range of 5 millimeters to 7 millimeters. The apparatus may provide live electrical feedback in response to a bipolar current from an electrode. The bipolar current may be in a range of 2 mA to 8 mA. The apparatus may include a device for creating an electric field around the wire.

In some aspects, the apparatus includes a probe such that the electric field from the wire induces a current in the probe. The current in the probe may be analyzed to determine an area of the body in which the probe is located. An indicator may be used for signaling when the probe is within a predetermined distance from the wire. In some aspects, the apparatus includes an inner conductor a first insulating layer surrounding the inner conductor, a conductive layer surrounding the first insulating layer, and a second insulating layer surrounding the conductive layer. In some aspects, the wire emulates a right arm corticospinal tract and the apparatus includes a second wire that emulates a right leg corticospinal tract. The second wire may be at least partially surrounded by the body. The apparatus may include a third wire that emulates a left leg corticospinal tract, that is at least partially surrounded by the body. The apparatus may include a fourth wire that emulates a left arm corticospinal tract, that is at least partially surrounded by the body.

In one aspect, a method is provided for creating a surgical training model. The method includes providing a wire that emulates a corticospinal tract. The method also includes providing a component that emulates a tumor and surrounding the wire and the component with a first section of a body that emulates brain tissue and a complementary second section of the body.

The some aspects, the first section of the body and the second section of the body and the component are 3D printed. The first section of the body and the second section of the body may comprise a polymeric material. The polymeric material may have an elasticity of 7000 kPa±440 kPa. The polymeric material may be selected from the group including polyvinyl alcohol, polylactic acid, silicones, thermoplastic elastomers, and mixtures and blends thereof. The first section of the body and the second section of the body may have a first color, the component may have a second color, and the first color and the second color may be different.

In one aspect, another method is provided for creating a surgical training model. The method includes creating a body that emulates brain tissue. The body includes a first interior space dimensioned to receive a wire that emulates a corticospinal tract and a second interior space dimensioned to receive a component that emulates a tumor. The first interior space and the second interior space are filled with a dissolvable material. The method also includes contacting the dissolvable material with a solvent to remove the dissolvable material from the first interior space and the second interior space. The method also includes positioning the wire in the first interior space; and positioning the component in the second interior space.

In some aspects, the body is 3D printed. The body may be formed of a polymeric material. The polymeric material may have an elasticity of 7000 kPa±440 kPa. The polymeric material may be selected from the group including polyvinyl alcohol, polylactic acid, silicones, thermoplastic elastomers, and mixtures and blends thereof. The dissolvable material may include a water soluble polymer including polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids, polyamides, polyacrylamide, copolymers of maleic/acrylic acids, polysaccharides, natural gums, and blends and mixtures thereof. The dissolvable material may be polyvinyl alcohol, and the solvent may be water.

In some aspects, The first section of the body and the second section of the body may have a first color, the component may have a second color, and the first color and the second color may be different.

In one aspect, another method is provided for creating a surgical training model. The method includes positioning a wire that emulates a corticospinal tract in a mold. The method also includes positioning a component that emulates a tumor in the mold. The method also includes placing a moldable material in the mold and allowing the moldable material to set to a body that emulates brain tissue, the body surrounding the wire and the component.

In some aspects, the mold may be formed of a dissolvable material, and the method may include contacting the mold with a solvent to remove the mold from the body. he body comprises a polymeric material. The polymeric material may have an elasticity of 7000 kPa±440 kPa. The polymeric material may be selected from polyvinyl alcohol, polylactic acid, silicones, thermoplastic elastomers, and mixtures and blends thereof. The dissolvable material may be formed from a water soluble polymer selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids, polyamides, polyacrylamide, copolymers of maleic/acrylic acids, polysaccharides, natural gums, and blends and mixtures thereof. The dissolvable material may include polyvinyl alcohol, and the solvent comprises water.

In some aspects, the first section of the body and the second section of the body have a first color, the component has a second color, and the first color and the second color are different.

These and other features, aspects, and advantages of the present invention will become better understood upon consideration of the following detailed description, drawings and appended claims.

### BRIEF DESCRIPTION OF THE DRAWINGS

FIG. 1 shows a non-limiting example method for replicating cortical mapping.

FIG. 2 shows a non-limiting example model that includes the corticospinal tract and the regions of the motor cortex that correlate with arm and leg movement.

FIG. 3 shows a flowchart for a non-limiting example method for use in simulating brain tracts.

FIG. 4 shows a non-limiting example electrical filament that may be used for simulating brain tracts in the model.

FIG. 5 shows a non-limiting example mechanism for feedback with LED's.

FIG. 6 shows a non-limiting example brain model with an inner representation of a tumor.

FIG. 7 shows a non-limiting example ink loaded 3D printed spherical tumor infused with a dye.

FIG. 8 shows a non-limiting example 3D printed brain model with two filaments.

FIG. 9 shows the tumor void depicted in FIG. 8 filled to form a simulated tumor.

FIG. 10 shows a non-limiting example of a molded model where the molded model may be 3D-printed from a water-soluble material, and a polymer may be poured in to form the brain shape.

FIG. 11 shows a non-limiting example functional model system.

Like reference numerals will be used to refer to like parts from Figure to Figure in the following description of the drawings.

### DETAILED DESCRIPTION OF THE INVENTION

Systems and methods are provided for a functional model of the brain to be used for training medical professionals, such as to resect brain tumors outside of the operating room. In some configurations, the model incorporates electrical feedback. Electrical feedback, or cortical mapping, is a component of all awake brain tumor resection surgeries and conventionally is only taught by observation. Many neurosurgical residents say that they learn this skill on the job, meaning that the most useful experience and knowledge that they gain about the surgical procedure happens live in the operating room with a patient relying on them to resect a tumor without leaving permanent deficiencies. This is not ideal, and having models that incorporate more aspects of a live surgery can aid in the prevention of this problem. These models may be used to change the way tumor resection techniques are taught to residents and allow them to train more safely and successfully.

Simulators have proven validity and demonstrable transfer of skills to the clinical setting. There is currently no commercialized surgical simulator of brain mapping; trainees are limited to learn by the classic teaching method of “see one, do one, and teach one,” instead of a more appropriate “see one, simulate one (or many), do one, and teach everyone.” The anatomy and nuances of brain mapping surgery can be simulated using current 3-dimensional (3D) printing techniques to replicate functional white matter tracts and cortex. In some configurations, an anatomically accurate and electrically responsive high-definition biomimetic 3D-printed tool may be used for training and simulating brain mapping.

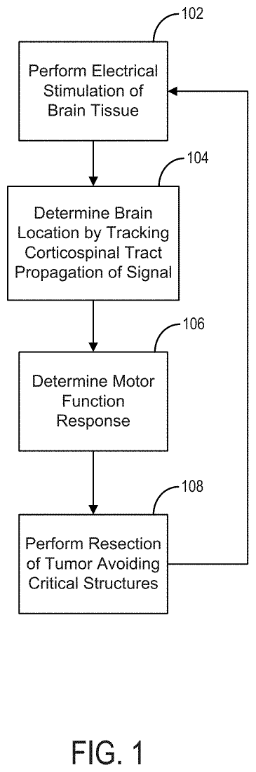

Referring to FIG. 1, a non-limiting example method is shown for replicating cortical mapping, which may use electrical stimulation during a procedure. The purpose of cortical mapping is to determine the areas of the brain in the patient that are vital to certain functions, such as motor and speech abilities. To perform cortical mapping, the surgeon touches the surface of the brain with a bipolar electrode and stimulates the nearby tissue at low voltages and currents, as shown at step 102. In a non-limiting example, the simulation may include 2-8 mA bipolar stimulation, in 5-10 sec. intervals with 3-10 mV. Since the brain is essentially a collection of circuits, inducing current into these circuits creates a response in the various circuit components, which in this case are neurons. The signal propagates through the cortical spinal tract, and the electrical responses may be used to inform the neurosurgeon of their relative location in the brain at step 104, The signal propagation may also inform how far away a neurosurgeon is from important bundles of axons that conduct electricity, called tracts. In some configurations, the surgeon can relate the voltage of applied stimulation to the distance they are from a critical tract. A motor function response may be determined at step 106. Returning to the circuit metaphor, the brain tissue between the electrode and the tract acts as a resistor. Thus, there is a linear relationship between the amount of tissue between the electrode and motor tract and the voltage needed to overcome this distance. The cortical mapping process may inform the surgeon how close they are to critical structures or important tracts as they cut around the tumor. The neurosurgeon may proceed with tumor resection in this manner that avoids a critical structure, such as eloquent brain tissue, at step 108. A model may be used to emulate this anatomical response, so that residents have the opportunity to learn and practice this technique outside the operating room.

A model of the brain for this type of procedure may be configured to interact with the other pieces of surgical equipment used. In a non-limiting example, the model may interface with the neurostimulator, which typically features a bipolar electrode that delivers stimulation to the surface of the model. In a non-limiting example, the model may interface with a wire probe that represents the neurostimulator but that does not deliver stimulation. In a non-limiting example, the model may interface with other handheld surgical devices such as the scalpel and suction device, which are used during surgeries to cut and clear tissue and liquids. The users may include neurosurgeons and neurosurgical residents. As far as design implications, these users are all highly educated and trained, but work in fast-paced, high pressure environments, and so the model may be configured to be accurate to how procedures occur in the operating room yet simple enough to learn easily.

TABLE 1
Non-limiting Example Design Inputs for a functional
brain model for surgical procedure training.

| Input | | | | |
|---|---|---|---|---|
| Description | User Needs | Design Inputs | Metric | |
| Physical | Anatomically | Brain weight | 1198-1336 | g |
| Embodiment | accurate | Brain volume | 1130-1260 | cm³ |
| | | Brain width | 140 mm within 5% | |
| | | Brain length | 167 mm within 5% | |
| | | Brain height | 93 mm within 5% | |
| | | Cerebral cortex | 1.5-4.5 | mm |
| | | thickness | | |
| | | Lobe as percent of | Frontal: 41%, | |
| | | total cerebral cortex | Temporal: 22%, | |
| | | volume | Parietal: 19%, | |
| | | | Occipital: 18%; | |
| | | | within 5%. | |
| | | Brain color | 663 C Pantone | |
| | | Corticospinal tract | 5-7 | mm |
| | | diameter | | |
| | | Corticospinal tract | x = −70 to 4, | |
| | | position | (right/left) | |
| | | | y = −43 to 7, | |
| | | | (rostral/caudal) | |
| | | | z = 19 to 76 | |
| | | | (dorsal/ventral) | |
| | Represents low- | Tumor diameter | 1-3 | cm |
| | grade glioma | Transition range | 3-5 | mm |
| | pathophysiology | diameter | | |
| | | Tumor color | 664 C Pantone | |
| | Touch | Elasticity | 7000 kPa ± 440 kPa | |
| Performance | Receives | Stimulation intensity | 2-8 mA, 3-10 mV | |
| | electrical | Stimulation duration | 5-10 | sec |
| | stimulation | Produces a linear | 1 mm = 1 mV ± 0.4 | |
| | | relationship between | | |
| | | distance and voltage | | |
| Shelf Life | Better shelf life | Shelf life | >6 | years |
| | than cadaver | Is stable at room | 25° | C. |
| | | temperature | | |
| Interfacing | Usable with | Model may function | input of 2-8 mA, | |
| Devices | other surgical | with stimulation from | 3-10 mV | |
| | equipment | bipolar electrode | | |
| | | Model may send | less than 20 mA | |
| | | sufficient signal to | | |
| | | turn on feedback | | |
| | | device | | |
| Human | Can be used to | Duration of single use | >1 | hour |
| Factors | train for longer | | | |
| | than one | | | |
| | procedure | | | |

A list of non-limiting example design inputs (e.g., electrical stimulation, elasticity, and tract representation) for a functional brain model sufficient for training purposes are shown in Table 1. These inputs were identified as significant needs for neurosurgeons and neurosurgery residents. A conventional model for training is only an anatomical representation of the brain, which provides no functionality or ability to practice neurosurgical techniques. The non-limiting design inputs in Table 1 may provide for a training device to make the training device brain feel and respond like real brain tissue during a procedure, such as tumor resection surgery, and the like.

In some configurations, design inputs include user experiences in current training methods, gaps of understanding, surprises upon entering the operating room, current neurosurgical procedures, the challenges of tumor resection, and the like. Design inputs may also be categorized into critical, expected, and wanted needs. Categorization may be further validated in order to exclude bias.

The design inputs in Table 1 may be summarized into four main user needs: anatomical accuracy, touch, stimulation feedback, and representation of low-grade glioma pathophysiology. Stimulation feedback includes where the model may respond to electrical stimulation and be able to convey this information back to the trainee. Design inputs may also take into account touch, such that the model may feel more similar to live brain tissue in bounciness and density than a cadaver in order to be useful.

In some configurations, human factor considerations may be included in the design inputs. Human factors include haptic feedback from cutting into the brain, replication of a surgical environment, collaboration with other professionals for signal interpretation, how the model will be held while in use, and the like. A successful model may allow for more accurate training than operation on a cadaver, such that the model may have better texture and interactive capabilities, which may be delineated in the design inputs.

Any appropriate material may be considered as part of the design inputs, and as the model may not be implanted in the body it may not need to be biocompatible. The materials for a device may be selected to not be made out of harmful materials, as the surgical trainees need to interact with it regularly. The model may include dissolvable components that may be dissolved with a solvent (e.g., water) from the mold to accommodate electrical tracts. The dissolvable material can be a water soluble polymer selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids, polyamides, polyacrylamide, copolymers of maleic/acrylic acids, polysaccharides, natural gums, and blends and mixtures thereof. The non-dissolvable component of the model may include polylactic acid, silicone, thermoplastic elastomers such as styrenic block copolymers, thermoplastic polyolefin elastomers, thermoplastic polyurethanes, thermoplastic copolyesters, thermoplastic polyamides, and mixtures thereof. The device may also not need to be sterilized to food or medical grade levels. The device may be designed to run on electricity provided by a standard American outlet or standard battery to increase accessibility and lower cost.

Design inputs may be categorized as: electrical stimulation, elasticity, and tract representation. The lack of live electrical feedback present in current surgical training methods presents a challenge for users. To address this need, the model may be able to respond to a bipolar current ranging between 2-8 mA, in a non-limiting example. The timing of the model response may be used to simulate the real-life surgical scenario. In a non-limiting example, the model may provide feedback within 10 seconds, as in a surgical procedure any stimulation longer than 10 seconds may result in seizure of the brain tissue. The model may also provide for the ability to train for longer than the duration of a normal procedure, such as longer than 30 minutes, in a non-limiting example. In another non-limiting example, the model may be able to operate for at least one hour. There is also a linear correlation between the distance between the electrode and the tract and the amount of voltage applied through the stimulator, which is quantified as 1 millimeter of tissue distance is roughly equal to 1 mV of applied voltage.

The texture of the model may be equal to or better than cadaver tissue. In order to quantify the feel of brain tissue, a measurement of elasticity may be performed, which is the ability of a material to resume its original shape after a stretch, compression, or other deformation is applied. The elasticity of brain cadaver tissue is 70,000±44,000 kPa, while the elasticity of live bovine tissue is 1.389 kPa±0.289 kPa. In a non-limiting example, in order to have the model feel more like live brain tissue, the model elasticity may be selected to be an order of magnitude less than the cadaver elasticity, with a two order of magnitude decrease in tolerance. In the non-limiting example model, the elasticity was 7000 kPa±440 kPa.

Design inputs for the model may include the incorporation of white matter tracts, which interconnect and convey information from the cerebral cortex to subcortical structures. These tracts begin in the motor cortex, stemming from the surface of the brain and travel within the brainstem to end in the spinal cord and interconnect with muscles and sensory terminals. In a non-limiting example, the motor tracts of the arm and leg may be included in the model.

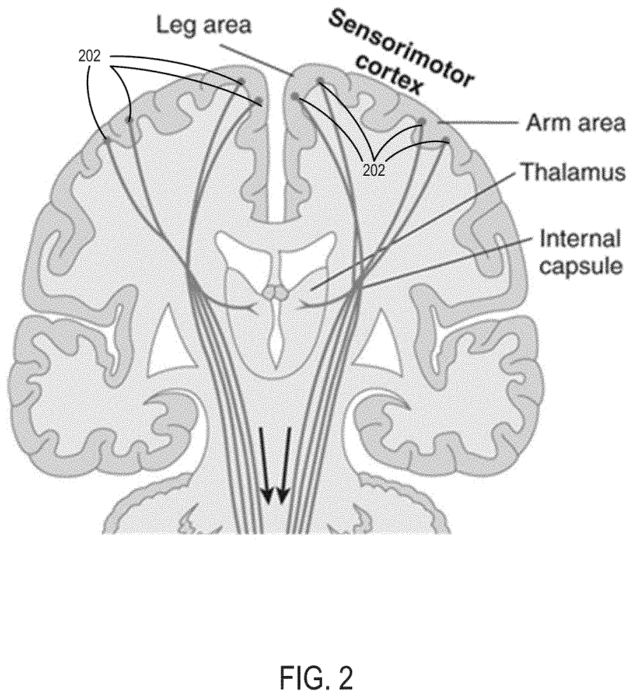

Referring to FIG. 2, the model may include the corticospinal tract and the regions of the motor cortex that correlate with arm and leg movement, as shown. In a non-limiting example, the three-dimensional location ranges of the tract start points are as follows: x=−70 to 4 (right to left), y=−43 to 7 (rostral/caudal), and z=19 to 76 (dorsal/ventral). This location may correspond to the dots 202 in FIG. 2. The tract end points may be at the most medial, rostral, and ventral part of the model. In a non-limiting example, the tract diameter may be between 5-7 millimeters.

The design inputs may include anatomical accuracy, tumor representation, and operational considerations. The model may be as anatomically accurate as possible, which is an advantage because the brain is very complex and a model that looks and feels similar to a human brain during an operation may provide superior training than the systems that are conventionally available. The average adult brain weighs 1198 grams for females and 1336 grams for males. These weights can fluctuate based off of various demographic factors, such as age, weight, and height. In a non-limiting example, the model weighs within the averages noted above, and the volume of the model may be from 1130-1260 cm³, also within the range of the female to male average. The average brain dimensions include a width of 140 millimeters, a length of 167 millimeters, and a height of 93 millimeters. In a non-limiting example, the device may have these dimensions within a tolerance of 5%. The thickness of the cerebral cortex is in a range of 1.5-4.5 millimeters. The brain model may be selected to be anatomically proportional. The brain is composed of four lobes, and these lobes may be configured to all be anatomically accurate within their size in relation to one another. These proportions are traditionally measured as a percentage of the total cerebral cortex volume; on average, the frontal lobe accounts for 41% of the cerebral cortex volume, the temporal lobe is 22%, the parietal lobe is 19%, and the occipital lobe is 18%. The color of healthy brain tissue is a Pantone code of 663 C. In some configurations, the model may be configured to reflect this color.

In some configurations, the model may contain a tumor for users to practice performing a resection. Safely resecting a tumor near or at an eloquent brain region is one of the most difficult parts of performing an intracranial surgery. In a non-limiting example, the model may contain a low-grade glioma, which is similar in structure and color to healthy brain tissue; just like healthy tissue, gliomas are soft, gray, and have a density and texture comparable to gelatin sold under the trademark Jell-O®. These similarities make it difficult to determine the boundaries of the tumor. To approximate this, in the non-limiting example, the model may have a 1-3 centimeter diameter spherical tumor that is a slightly darker gray (Pantone 664 C) than the surrounding tissue (Pantone 663 C), as well as 3-5 millimeter sphere of transition space around the tumor. This is the average size of tumors that are removed via resection surgery. The transition space is healthy, noncancerous tissue that has been impacted by tumor growth via cell crowding and angiogenesis, which is usually a finding with higher-grade tumors.

The design inputs may include the model's shelf life, human factors, and regulatory considerations. The current gold standard for training is cadavers, which, when embalmed, last for up to six years. In some configurations, the model is configured to have a greater shelf life than the current gold standard. In a non-limiting example, the model may last for over six years and remain stable at room temperature for storage purposes. Since the models may be used by neurosurgeons and neurosurgery residency programs; the models may be stored in labs and classrooms, and therefore will be kept at 25° C., for an unknown period of time. The model may be used in a classroom or clinical environment; with the users including neurosurgery residents and neurosurgeons training for tumor resection surgery. The model may also be used in clean lab spaces. The model may be used during lab or class sessions, so the user may be surrounded by other students working on their own models.

In some configurations, the model may be used with a stimulator as in current surgical procedure. The model may receive the stimulation from the neurostimulator, integrate it, and output it to the users for feedback. In a non-limiting example, this output may include turning on an LED (Light Emitting Diode) if a sufficient amount of voltage is received, such as an output current no more than 20 mA. The model may be engineered in a way where significant harm to the user is avoided.

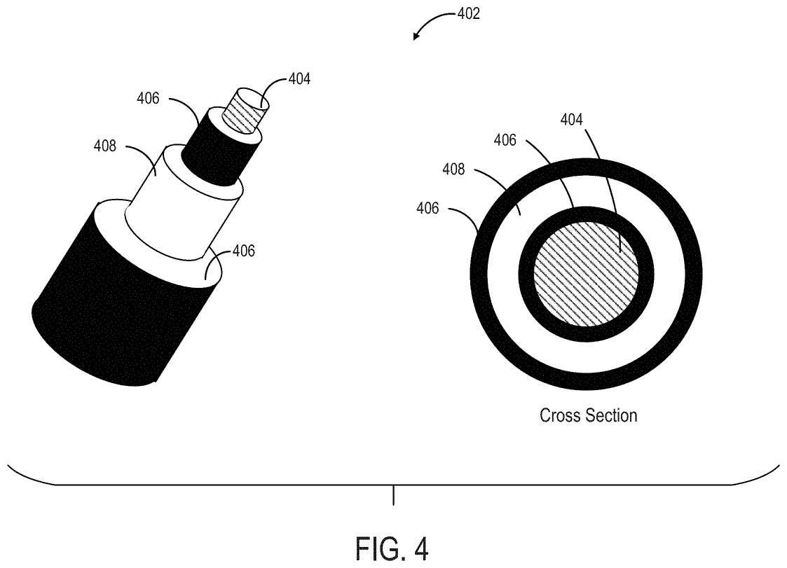

Referring to FIG. 4, a non-limiting example electrical filament 402 is shown that may be used for simulating brain corticospinal tracts in the model. A functional brain model includes a set of circuits that are representative of the conductive brain pathways in the motor system, called white matter tracts. The tracts may be modeled as a combination of both wires and 3D-printed filament. The inner portion of the tracts may be a conductive wire-like component 404 that, if severed, would signal to the trainee that they caused irreparable damage to the patient. The second layer 408, or outer ring of the cross-section, may also be conductive, but insulated from the inner layer by insulation layers 406. This second layer 408 serves as a warning layer, providing the trainee with feedback that they are nearing an important tract. Insulation layers 406 are not conductive, while wire component 404 and warning layer 408 are conductive.

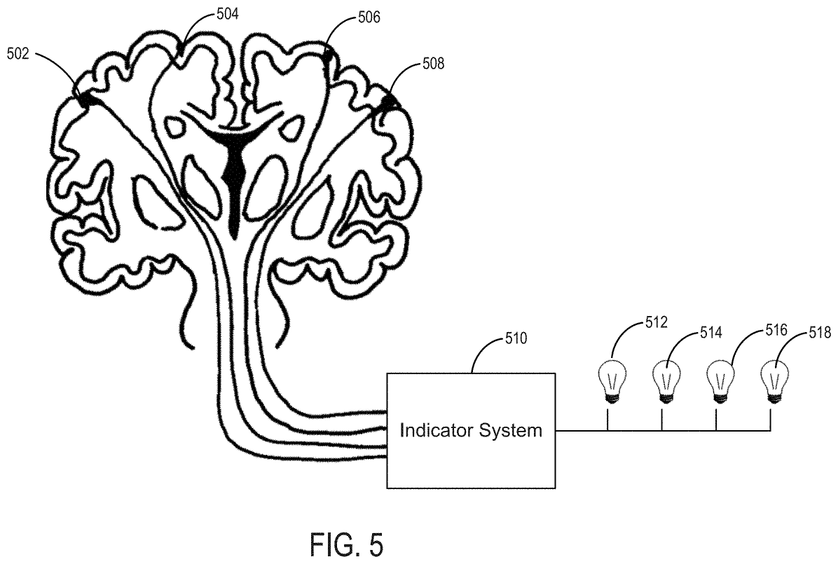

Referring to FIG. 5, a non-limiting example mechanism for feedback is shown with LED's 512, 514, 516, and 518. The dots shown on the image of a brain indicate the starting point of each tract. In particular, left leg tract 502 is linked through the indicator system 510 to left leg LED 518. Left arm tract 504 is linked through the indicator system 510 to left arm LED 516. Right leg tract 506 is linked through the indicator system 510 to right leg LED 514. Right arm tract 508 is linked through the indicator system 510 to right arm LED 512. Stimulation occurs at these points or along the connecting circuit, and feedback may be given by the illumination of the corresponding LED. This feedback mechanism shows the trainee whether stimulation of a certain tract has occurred.

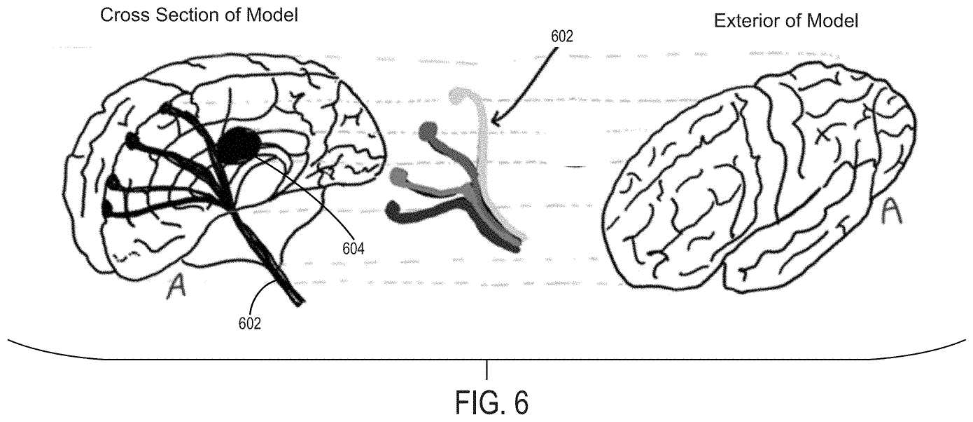

Referring to FIG. 6, a non-limiting example brain model is shown with an inner representation of a tumor. The model is shown in exterior view and in cross section that has been drawn vertically through the model with plane “A.” The model may include a 3D printed modular design. The modular design may include two modular halves of the brain that will encapsulate the motor tracts 602 (four as shown in the example) and a spherical tumor 604. The tumor 604 may be represented by a 3D-printed inclusion, such as the sphere shown measuring 1-3 centimeters in diameter. In order to distinguish this tumor tissue from the surrounding tissue, the sphere may be loaded with a dyed substance.

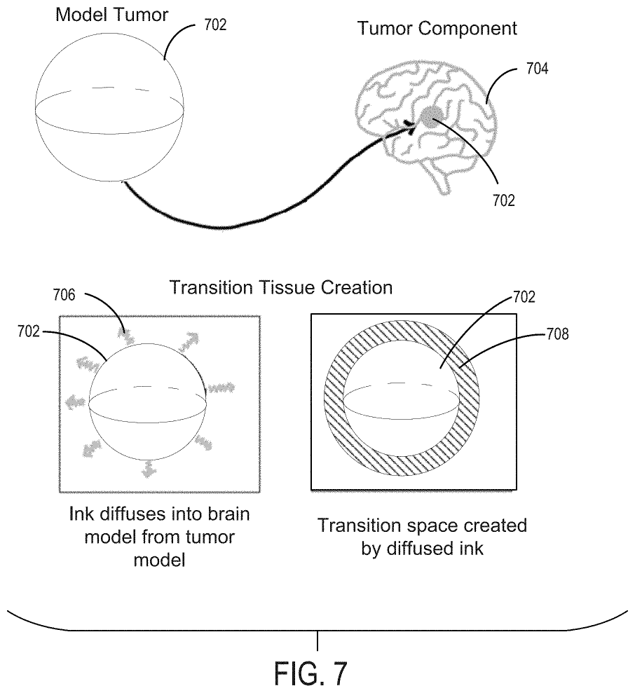

Referring to FIG. 7, a non-limiting example ink loaded 3D printed spherical tumor 702 is shown infused with a dye 706. The dye 706 may diffuse radially outward from the tumor 702, creating a color gradient indicative of the transition space 708 between tumor 702 and healthy tissue. In a non-limiting example, the dye 706 may diffuse slowly out from the tumor 702, creating a 3-5 millimeter transition space 708 color gradient to simulate a transition between the represented healthy and tumor tissues.

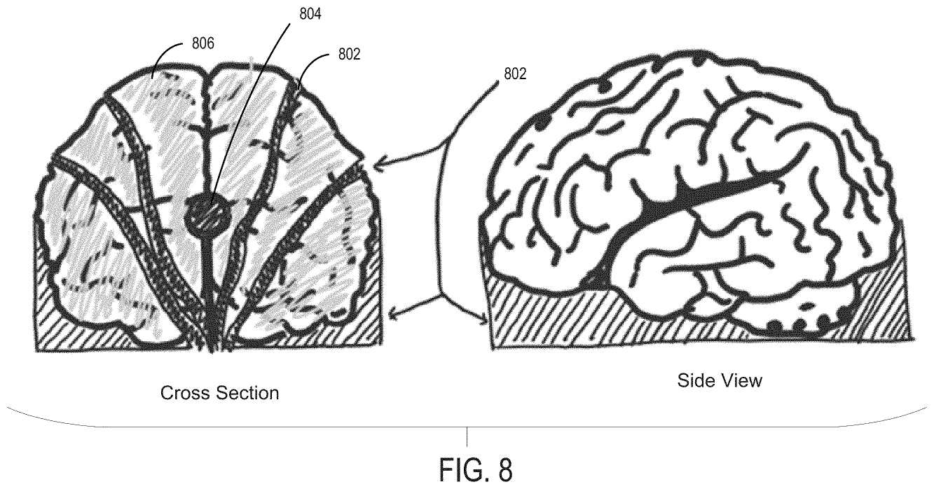

Referring to FIG. 8, a non-limiting example 3D printed brain model is shown with two filaments. The filaments may be a poly-vinyl alcohol (PVA) and/or a PVA/polylactic acid (PLA) blend. The PVA scaffold 802 is shown by the lines in the cross-section, and the PVA/PLA blend 806 is shown in the bulk of the model. The PVA in this non-limiting example served as a structural filler which can be dissolved in water to create gaps for both the tracts and the tumor 804 on the interior. Due to spacing restrictions, the tumor 804 in this model may be represented by another polymer that can be poured into the hole left by the dissolved PVA. The tumor 804 placement depicted in the figures is for example purposes only. It can be appreciated that the tumor 804 may not be placed between the two hemispheres, but instead on the superolateral surface of the brain, in another non-limiting example.

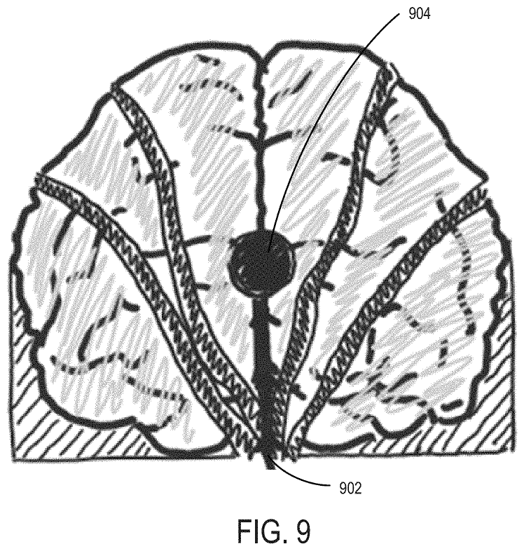

Referring to FIG. 9, the tumor void 902 depicted in FIG. 8 may be filled to form a simulated tumor 904. Due to the dissolution of the PVA by water, a large gap to insert a spherical tumor does not exist, as indicated in FIG. 8. Instead, a flexible polymer may be poured into the model and fill the spherical gap or tumor void 902 where the tumor 904 will be represented.

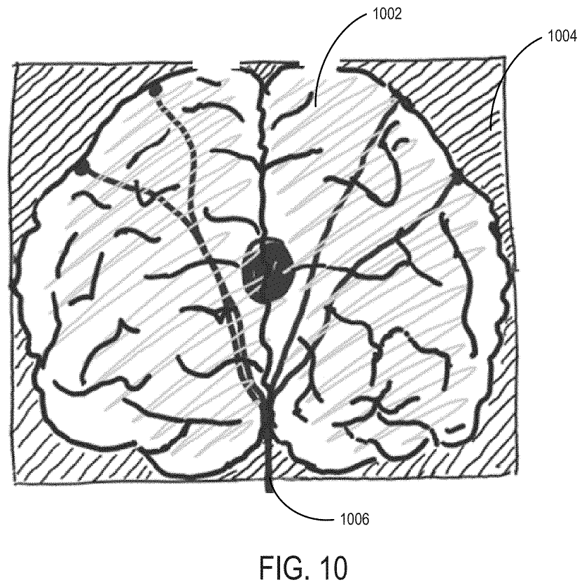

Referring to FIG. 10, a non-limiting example of a molded model is shown. The molded model may be 3D-printed from water-soluble PVA, and a polymer 1002 (e.g., silicone) may be poured in to form the brain shape. The entire structure may be placed in water and the PVA mold 1004 may dissolve away allowing for the gyri and sulci to maintain their shape. As shown, the molded model may allow for placement of the combination tract 1006 on the outside of the brain as well as the ink-loaded tumor placement within the brain. The material with which the mold will be filled may need to meet similar elasticity to the 3D printed models.

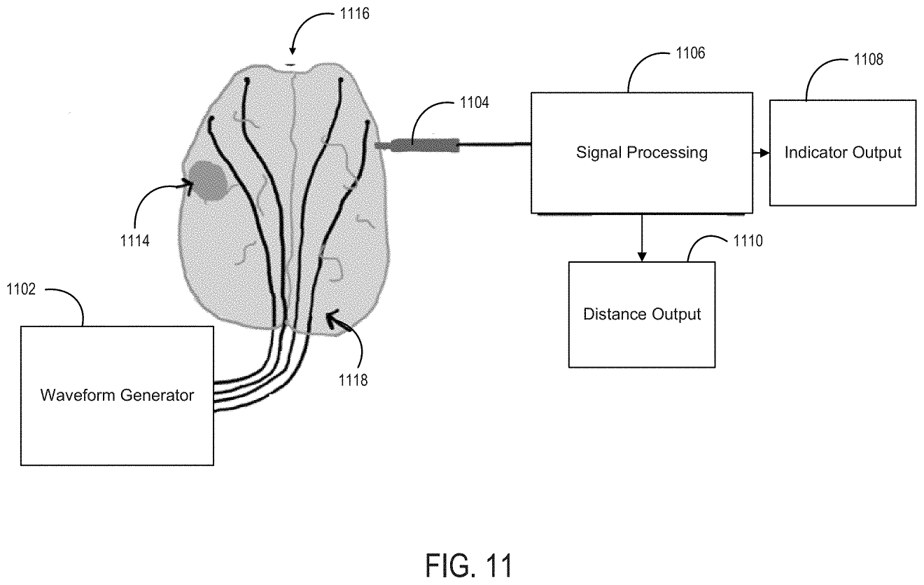

Referring to FIG. 11, a non-limiting example functional model system is shown. The functional model system includes a model component 1116 with a simulated tumor inclusion 1114. A waveform generator 1102 may be used to provide electrical signal to the wires 1118 embedded in the model 1116. An electrical probe 1104 may be used with a signal processing system 1106, which may illuminate an indicator 1108, such as an LED, when the distance output 1110 detects electrical signal at a threshold value.

The following Example has been presented in order to further illustrate the invention and is not intended to limit the invention in any way.

A surgical training model apparatus according to an example embodiment of the invention can comprise a polyvinyl alcohol (PVA) mold. The mold may be configured using a 3D printer, such as in a Makerbot Method, a slush mold, or any appropriate method. The model itself may be made of a silicone. In a non-limiting example, the silicone is EcoFlex 00-20 material with wires inside of it and a silicone tumor. Silicone dyes may be used to color both the model and/or the tumor. In some configurations, a combination of a pink and grey color may be used with the tumor being slightly darker than the overall model.

The PVA mold may be an inverse of patient brain imaging created in a program, such as 3D Slicer, that allows for MRI imaging to become an 3D printer compatible file. The model may be patient specific and may be made from patient imaging. The mold may be printed in two pieces to be placed together. The mold may be placed together and filled with the uncured dyed silicone, the wires (to serve as corticospinal tracts in the model), and a dyed solidly cured silicone tumor. The wires and the tumor may be held in place using any appropriate mechanism to stay in the correct position while the silicone cures and sets. In some configurations, the PVA mold may be placed in water in order to form the final silicone model with the proper surface including well-formed sulci. When placed in water, the PVA may dissolve in the water solvent and leave the final silicone model behind. The inserted wires may then be connected to the electrical component. A neurosurgeon can then use the model to practice electrical stimulation and resecting a tumor. The model may also be used for pre-operative planning due to patient-specific imaging.

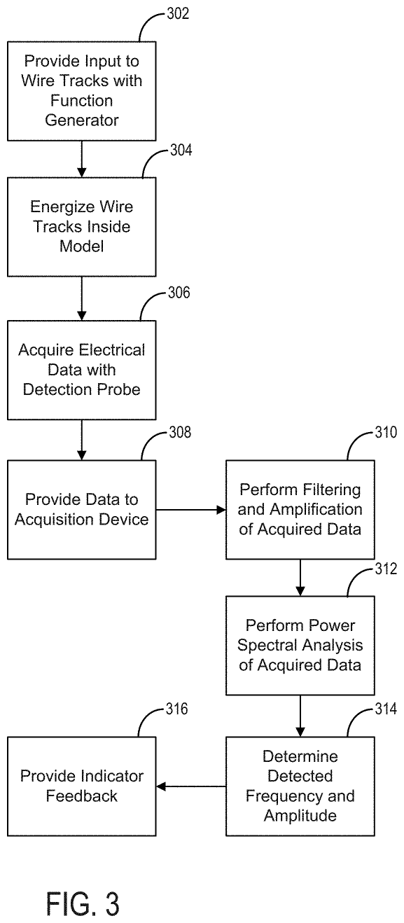

Referring to FIG. 3, a flowchart of a non-limiting example method is shown for the electrical component of the model. The electrical portion of the model may include the wires running through the model (e.g., tracts), and the probe. In a non-limiting example, the tracts include four wires. A waveform generator may be used to send a waveform through the tracts to create an electric field around the wire at step 302. This electric field can be made larger or smaller depending on the waveform characteristics. In this way, the tracts may be energized at step 304, and electrical data may be acquired with a probe at step 306. The probe may be made of a conductive coiled or uncoiled wire. The data may be provided to an acquisition device at step 308. As the probe approaches the tract, the electric field from the tract induces a current in the probe, which may then amplified, filtered, and analyzed at step 310 to determine proximity to the corticospinal tracts. Any number of electrical tracts may be used in the model, including tracts that mimic specific anatomical configurations, such as speech and motor tracts. Example circuit components include filters and amplifiers and may also be represented in a digital format through analytic software (e.g. MATLAB, LabView, and the like). A power spectral analysis may be performed on the acquired data at step 312, and a frequency or amplitude of the electric field may be determined at step 314. An indicator, such as an LED, may provide feedback to the user at step 316, such as whether the user has come in contact with a tract, or the location of the tract relative to the user, and the like.

A tumor may be represented as a sphere, such as a sphere made of the same molded silicone as the brain. The tumor may be colored slightly darker than the model. In a non-limiting example, the tumor may be a sphere 3-5 millimeters in diameter set in a simulated brain model.

In some configurations, the systems and methods of the current disclosure provide an improved surgical training model apparatus and methods for creating a surgical training model apparatus.

Non-Limiting Example Brain Model

Thirteen neurosurgeons and neurosurgery residents were interviewed in an open-ended format to determine critical design aspects and educational value of a 3D-printed training model. Anonymized brain magnetic resonance imaging (MRI) data were converted into a 2-piece inverse 3D rendered shell, which was printed from polyvinyl acetate (PVA) using MakerBot method 3D printers (MakerBot, New York City, USA, 2019). After printing, the two pieces of the shell were attached, forming an anatomically accurate mold of the brain. Then, a preset silicone molded tumor dyed with the FUSEFX BC-03 dye was inserted into the full brain mold in the superolateral surface in the posterior end of the middle frontal gyrus anterior to the motor cortex. The tumor was further dyed with a layer of blue silicone pigment for a more life-like grayish appearance. The shell was filled with Ecoflex-20 silicone by Smooth-On and dyed with equal parts of the FUSEFX silicone dyes S-301-D and BC-03 to create the opaque pink color. Once the silicone cured for 12 hours, the silicone-filled shell was completely submerged in water to dissolve the PVA mold.

Functional MRI and diffusion tensor imaging (DTI) data were analyzed in the open-source software 3D Slicer15 (Slicer 4.10.2, www.slicer.org, 2019) to generate tractography. This was used to guide wire placement in the created mold, to approximate the projection fibers from the arm and leg areas in the motor homunculus that should be mapped and preserved during surgery. The wires generated electric fields that were detected by a handheld probe through electrical coupling. The handheld probe included a shielded wire connected to a data acquisition (DAQ) system (myDAQ 19.0.0, National Instruments, Austin, USA, 2019). The electric fields were generated digitally through MATLAB (MATLAB R2020b, The MathWorks, Inc., Natick, USA, 2020) with a 2Vpp square wave running through each tract, at frequencies of 10 and 20 kHz, respectively. Electrical parameters remained constant in both cortical and subcortical regions of the tract. Collected raw data pass from the DAQ device through processing software written in LabVIEW (LabVIEW 2020, National Instruments, Austin, USA, 2020). Processing included a bandpass filter between 5 and 20 kHz, amplification with a gain of 30, and spectral power analysis to discriminate between the 2 tracts and to predict relative location.

The relationship between the electrical signal acquired and the distance between the probe and the simulated tract was quantified (n=3 trials) by measuring the response recorded by the simulator between 0 and 3 cm at increments of 0.5 cm using a standard ruler. A baseline reading was taken at a distance greater than 30 cm from the model for calibration purposes.

The elasticity, in kilopascals (kPa), was determined for human tissue, as well as animal tissues from Rhesus monkeys, rodents, cows, and pigs. These values were then compared to the elasticity of Ecoflex-20 silicone to demonstrate material accuracy. A follow-up survey was conducted in Likert format among 32 neurosurgical professionals in order to assess the model's validity and applicability to surgical and educational scenarios.

Electrical data were assessed with a linear regression and estimation of fit. A 95% CI based on observed variance was calculated to ensure that no outliers were present. Material results were reported as percent improvement over baseline, as results were compared to values from literature.

Neurosurgeon interviews and design ideation via multiple interviews (n=13) with neurosurgeons who routinely perform awake and asleep craniotomies for brain tumor resection, primary needs were identified to guide the design of the simulator: anatomic accuracy (n=8/13, 61.5%), tactile feedback (n=7/13, 54%), and stimulation feedback (n=7/13, 54%). Emphasis was on creating a simulator that would replicate not only the brain anatomy and elasticity, but also the brain's electrical conductivity (n=13/13, 100%).

The final brain model used 540 g of PVA, contained 810 g of silicone, and measured 16×12×10 cm. This simulator allowed for replication of various surgical scenarios and training performance of microsurgical techniques including brain mapping and tumor resection. The simulator can be used for mapping functional cortex and subcortical regions adjacent to a glioma to perform safe resection.

The linear relationship between distance and amplitude for the electrical response was determined to be an acceptable representation of the properties of live brain tissue. A linear regression was performed on the data (n=3 trials), showing an r2=0.86. Additionally, almost all data (n=25/27, 93%) fell within the 95% CI that was calculated from observed data, indicating that there were few outliers, and that the device produced consistent results.

Brain tissue hardens postmortem. The elastic modulus of a fixed Rhesus monkey brain measures up to 140 kPa, 17 compared to the approximate elasticity of live brain tissue, which is reported between ˜1.9 and 60 kPa. 17-21 The silicone brain simulation model had an elasticity of only 55 kPa, which is within the range of reported biological values and over 60% closer to the reported average of 14.8 kPa when compared to cadaveric brain tissue.

The model may be used in educational and surgical practice environments. Other aspects of the device may include anatomic accuracy, material accuracy, and mimicry of real surgical scenarios.

Cortical mapping for safe intraoperative monitoring of brain function is the gold standard management for many neurological conditions, including brain tumors, epilepsy, selective vascular lesions (cavernomas, aneurysms, and arteriovenous malformations), and intraparenchymal hematomas. Mastering brain mapping requires extensive practice as a neurosurgeon and as a multidisciplinary team including neurosurgeons, neurologists, neurophysiologists, anesthesiologists, and the like. An anatomically accurate, electrically responsive, 3D-printed model to simulate the essential steps of performing a brain mapping operation may provide for enhanced training of the entire surgical team by replicating a multitude of surgical scenarios and improving interdisciplinary communication.

Previous conventional models have failed to simulate direct stimulation and electrical conduction, allowing for delineation of functional cortex and subcortical tracts. The consistent linear relationship that was quantified allows for the device to give an accurate prediction of nearby corticospinal tracts. The model may be used for simulating a live neurosurgery, without the use of actual human tissue. The simulator simulates the brain's normal anatomy and reproduces a similar elasticity. A patient-specific 3D-printed physical model can be conveniently created at a low cost for preoperative planning, procedure rehearsal, patient education, and resident training and evaluation, and the like.

The model may be incorporated into the resident surgical skills curriculum and used to evaluate trainee performance with quantifiable milestones to certify their competency prior to a real case. This may translate to more resident autonomy in the operating room (OR), improved surgical precision, and increased patient safety.

A functional patient-specific 3D-printed model may be superior to cadaveric dissection for simulating the resection of intraparenchymal brain tumors. Cadaveric tissue is unable to retain the material and electrical properties of the live brain. The ethical, religious, and pricing concerns of cadavers make them an inaccessible resource for many. As has been seen during the COVID-19 pandemic, external forces may reduce access to cadavers and limit the ability of trainees to travel for cadaver courses. A 3D-printed simulator can be transported easily and safely, and used in a variety of environments, while a cadaver's usage is restricted to laboratory space and storage. The lack of biological tissue in the simulator also may eliminate the need for sterilization of tools, personal protective equipment, and cleaning. The cost for the nonreusable components of the model may be minor.

3D printing technologies are increasingly used for neurosurgical simulation, ranging from simulators of superficial extra-axial and intra-axial tumor resection to deep intraventricular neuro-endoscopic tumor resection. These previous methods have failed to provide a functional anatomic model that allows stimulation of brain tracts not available in even the best cadaveric specimens. Virtual reality (VR) has also been used previously in surgical simulation, such as in laparoscopic and general surgery, but current VR technologies cannot accurately replicate the experience of complex microsurgical maneuvers unique to neurosurgery or the neural tissue's haptic feedback or response to surgical manipulation and tension forces.

In some configurations, other relevant white matter tracts may be used to increase the validity and applications of the model, such as white matter fiber tracts and connectomic networks that are relevant to mastering the cortical mapping technique. In some configurations, the model may include enhanced anatomic validity, tactile feedback, and approximation of live brain tissue during surgery. Patient-specific tractography may be merged with the silicone model at the time of creation to account for the effect of cerebral edema on nearby tracts. A conductive and malleable material, rather than a single wire, could be placed into the 3D-printed shell before the silicone molding process in order to integrate biomimetic tracts and improve their material properties.

In some configurations, real-time simulated electroencephalography or electrocorticography may be used to monitor electrical discharges and epileptiform activity while a surgical trainee is stimulating the model. Blood and cerebrospinal fluid circulation may be incorporated using mechanical pumps, which may replicate intraoperative complications such as vessel injury and seizures.

Feedback mechanisms may also help to better simulate the conditions of the operating room (OR). In some configurations, the use of audio feedback (in addition to visual) during stimulation may more accurately replicate the verbal feedback between neurosurgeon and neuropsychologist intraoperatively. Patient-specific models with different lesion types and locations can be manufactured and used for preoperative planning and to rehearse patient positioning and intraoperative team dynamics and OR setup, which may be beneficial especially for crowded ORs during awake craniotomies. An additional feedback metric could include amplitude and distance cutoffs for stimulation, in order to avoid seizures and tissue damage. The simulator could be configured to include fiducial markers to function with existing neuro-navigation systems.

In some configurations, a biomimetic 3D-printed model for simulating craniotomies and brain mapping may be used for resident learning, and for improving neurosurgical training methods. The realistic neural properties of the simulator may improve representation of a live surgical environment. Complicated tractography, blood and cerebrospinal fluid circulation, and feedback mechanisms may be conveyed in the simulator model. The model may enhance training of not only the neurosurgical trainee but also the entire surgical team by replicating a multitude of surgical scenarios and improving interdisciplinary communication and preoperative planning.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

### REFERENCES

1. Dewan M C, Rattani A, Fieggen G, et al. Global neurosurgery: the current capacity and deficit in the provision of essential neurosurgical care. Executive Summary of the Global Neurosurgery Initiative at the Program in Global Surgery and Social Change [published online ahead of print, 2018 Apr. 1]. J Neurosurg. 2018; 1-10. doi:10.3171/2017.11.JNS171500
2. Blakeslee, S., Broder, J. M., & Pollack, A. (2004, March 12). In Science's Name, Lucrative Trade in Body Parts. Retrieved from https://www.nytimes.com/2004/03/12/us/in-science-s-name-lucrative-trade-in-body-parts.html
3. Kenning T. Neurosurgical Workforce Shortage The Effect of Subspecialization and a Case for Shortening Residency. AANS Neurosurgeon. 2016; 25(4).
4. Yaeger K A, Munich S A, Byrne R W, Germano I M. Trends in United States neurosurgery residency education and training over the last decade (2009-2019). Neurosurgical Focus. 2020; 48(3).
5. Quinones-Hinojosa, Alfredo, 9/12/2020, Faith Colaguori, Rebecca Forry, Maité Marin-Mera, and Megan McDonnell, Neurosurgery techniques and experiences.
6. ReFaey, Karim, 9/12/2020, Megan McDonnell and Faith Colaguori, Neurosurgery techniques and experiences
7. Meyer, Frederic, 9/10/2020, Maité Marin-Mera and Rebecca Forry, Neurosurgery techniques and experiences.
8. Parney, Ian, 9/8/2020, Maité Marin-Mera and Rebecca Forry, Neurosurgery techniques and experiences.
9. Zwirner, J., Scholze, M., Waddell, J. N. et al. Mechanical Properties of Human Dura Mater in Tension—An Analysis at an Age Range of 2 to 94 Years. Sci Rep 9, 16655 (2019). https://doi.org/10.1038/s41598-019-52836-9
10. Budday S, Nay R, de Rooij R, et al. Mechanical properties of gray and white matter brain tissue by indentation. J Mech Behav Biomed Mater. 2015; 46:318-330. doi:10.1016/j.jmbbm.2015.02.024
11. Mayka M A, Corcos D M, Leurgans S E, Vaillancourt D E. Three-dimensional locations and boundaries of motor and premotor cortices as defined by functional brain imaging: a meta-analysis. Neuroimage. 2006; 31(4):1453-1474. doi:10.1016/j.neuroimage.2006.02.004
12. Sullivan E V, Zahr N M, Rohlfing T, Pfefferbaum A. Fiber tracking functionally distinct components of the internal capsule. Neuropsychologia. 2010; 48(14):4155-4163. doi:10.1016/j.neuropsychologia.2010.10.023
13. Clinical Neuroanatomy. In: Movement Co, ed. 28 ed.
14. Hardy W N, Mason M J, Foster C D, et al. A study of the response of the human cadaver head to impact. Stapp Car Crash J. 2007; 51:17-80.
15. Sultan, F. and Braitenberg, V. Shapes and sizes of different mammalian cerebella. A study in quantitative comparative neuroanatomy. Hirnforsch. 1993; 34:79-92
16. Miller, A. K., Alston, R. L., and Corsellis, J. A. Variation with age in the volumes of grey and white matter in the cerebral hemispheres of man: measurements with an image analyser, Neuropathol Appl Neurobiol. 1980; 6:119-132
17. Halle M, Tabs F, Kikinis R, Pieper S, Aucoin N. Generic Anatomy Colors. 3D Slicer. https://www.slicer.org/wiki/Slicer3:2010_GenericAnatomyColors. Published Aug. 27, 2012. Accessed Sep. 22, 2020.
18. McCall M. The Secret Lives of Cadavers. National Geographic 2016.
19. Blakeslee, S., Broder, J. M., & Pollack, A. In Science's Name, Lucrative Trade in Body Parts. New York Times. 2004 Retrieved from https://www.nytimes.com/2004/03/12/us/in-science-s-name-lucrative-trade-in-body-parts.html
20. GTSimulators.com. (n.d.). Retrieved from https://www.gtsimulators.com/Human-Brain-Models-s/6.htm

The citation of any document is not to be construed as an admission that it is prior art with respect to the present invention.

What is claimed is:

1. A surgical training model apparatus comprising:
a body that emulates brain tissue; and
a wire that emulates a corticospinal tract, the wire being at least partially surrounded by the body.

2. The apparatus of claim 1 further comprising:
a component that emulates a tumor, the component being at least partially surrounded by the body.

3. The apparatus of claim 2 wherein:
the body has a first color,
the component has a second color, and
the first color and the second color are different.

4. The apparatus of claim 3 wherein:
the body has a transition region adjacent the component, the transition region having a color gradient between the first color and the second color.

5. The apparatus of claim 1 wherein:
the body comprises a polymeric material.

6. The apparatus of claim 5 wherein:
the polymeric material has an elasticity of 7000 kPa±440 kPa.

7. The apparatus of claim 5 wherein:
the polymeric material is selected from the group consisting of polyvinyl alcohol, polylactic acid, silicones, thermoplastic elastomers, and mixtures and blends thereof.

8. The apparatus of claim 1 wherein:
the wire has a diameter in a range of 5 millimeters to 7 millimeters.

9. The apparatus of claim 1 wherein:
the apparatus provides live electrical feedback in response to a bipolar current from an electrode.

10. The apparatus of claim 9 wherein:
the bipolar current is in a range of 2 mA to 8 mA.

11. The apparatus of claim 1 further comprising:
a device for creating an electric field around the wire.

12. The apparatus of claim 1 further comprising:
a probe,
wherein the electric field from the wire induces a current in the probe.

13. The apparatus of claim 12 wherein:
the current in the probe is analyzed to determine an area of the body in which the probe is located.

14. The apparatus of claim 12 further comprising:
an indicator for signaling when the probe is within a predetermined distance from the wire.

15. The apparatus of claim 1 wherein the wire comprises:
an inner conductor,
a first insulating layer surrounding the inner conductor,
a conductive layer surrounding the first insulating layer, and
a second insulating layer surrounding the conductive layer.

16. The apparatus of claim 1 wherein:
the wire emulates a right arm corticospinal tract;
the apparatus further comprises a second wire that emulates a right leg corticospinal tract, the second wire being at least partially surrounded by the body;
the apparatus further comprises a third wire that emulates a left leg corticospinal tract, the third wire being at least partially surrounded by the body; and
the apparatus further comprises a fourth wire that emulates a left arm corticospinal tract, the fourth wire being at least partially surrounded by the body.

17. A method for creating a surgical training model, the method comprising:
(a) providing a wire that emulates a corticospinal tract;
(b) providing a component that emulates a tumor; and
(c) surrounding the wire and the component with a first section of a body that emulates brain tissue and a complementary second section of the body.

18. The method of claim 17 wherein:
the first section of the body and the second section of the body and the component are 3D printed.

19. The method of claim 17 wherein:
the first section of the body and the second section of the body comprise a polymeric material.

20. The method of claim 19 wherein:
the polymeric material has an elasticity of 7000 kPa±440 kPa.

21. The method of claim 19 wherein:
the polymeric material is selected from the group consisting of polyvinyl alcohol, polylactic acid, silicones, thermoplastic elastomers, and mixtures and blends thereof.

22. The method of claim 17 wherein:
the first section of the body and the second section of the body have a first color,
the component has a second color, and
the first color and the second color are different.

23. A method for creating a surgical training model, the method comprising:
(a) creating a body that emulates brain tissue, the body including a first interior space dimensioned to receive a wire that emulates a corticospinal tract and a second interior space dimensioned to receive a component that emulates a tumor, the first interior space and the second interior space being filled with a dissolvable material;
(b) contacting the dissolvable material with a solvent to remove the dissolvable material from the first interior space and the second interior space;
(c) positioning the wire in the first interior space; and
(d) positioning the component in the second interior space.

24. The method of claim 23 wherein:
the body is 3D printed.

25. The method of claim 23 wherein:
the body comprises a polymeric material.

26. The method of claim 25 wherein:
the polymeric material has an elasticity of 7000 kPa±440 kPa.

27. The method of claim 25 wherein:
the polymeric material is selected from the group consisting of polyvinyl alcohol, polylactic acid, silicones, thermoplastic elastomers, and mixtures and blends thereof.

28. The method of claim 25 wherein:
the dissolvable material comprises a water soluble polymer selected from the group consisting of polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids, polyamides, polyacrylamide, copolymers of maleic/acrylic acids, polysaccharides, natural gums, and blends and mixtures thereof.

29. The method of claim 25 wherein:
the dissolvable material comprises polyvinyl alcohol, and
the solvent comprises water.

30. The method of claim 23 wherein:
the first section of the body and the second section of the body have a first color,
the component has a second color, and
the first color and the second color are different.

31. A method for creating a surgical training model, the method comprising:
(a) positioning a wire that emulates a corticospinal tract in a mold;
(b) positioning a component that emulates a tumor in the mold;
(c) placing a moldable material in the mold and allowing the moldable material to set to a body that emulates brain tissue, the body surrounding the wire and the component.

32. The method of claim 31 wherein:
the mold comprises a dissolvable material, and
the method further comprises contacting the mold with a solvent to remove the mold from the body.

33. The method of claim 32 wherein:
the body comprises a polymeric material.

34. The method of claim 33 wherein:
the polymeric material has an elasticity of 7000 kPa±440 kPa.

35. The method of claim 33 wherein:
the polymeric material is selected from the group consisting of polyvinyl alcohol, polylactic acid, silicones, thermoplastic elastomers, and mixtures and blends thereof.

36. The method of claim 32 wherein:
the dissolvable material comprises a water soluble polymer selected from the group consisting of polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids, polyamides, polyacrylamide, copolymers of maleic/acrylic acids, polysaccharides, natural gums, and blends and mixtures thereof.

37. The method of claim 32 wherein:
the dissolvable material comprises polyvinyl alcohol, and
the solvent comprises water.

38. The method of claim 30 wherein:
the first section of the body and the second section of the body have a first color,
the component has a second color, and
the first color and the second color are different.
